(19) United States
(12) Patent Application Publication
(10) Pub. No.: US 2010/0168619 A1
(43) Pub. Date: Jul. 1, 2010

(54) COMBINATION WIRE GUIDE AND METHOD OF USE THEREOF

(75) Inventor: James C. Elsesser, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/344,888

(22) Filed: Dec. 29, 2008

(51) Int. Cl. A61M 25/00

(52) U.S. Cl. 600/585

(57) ABSTRACT

The present embodiments generally relate to a medical surgical device and specifically a wire guide for percutaneous placement within a body cavity. The wire guide includes a multi-filar coil having an increasing pitch towards the distal end of the wire guide. Methods of using the device are also provided.

### TECHNICAL FIELD

The present invention generally relates to a medical surgical device and specifically a wire guide for percutaneous placement within a body cavity. The flexibility of the wire guide varies along the length of the wire guide.

### BACKGROUND

Wire guides are commonly used in vascular procedures, such as angioplasty procedures, diagnostic and interventional procedures, percutaneous access procedures, or radiological and neuroradiological procedures in general, to introduce a wide variety of medical devices into the vascular system. For example, wire guides are used for advancing intraluminal devices such as stent delivery catheters, balloon dilation catheters, atherectomy catheters, and the like within body lumens. Typically, the wire guide is positioned inside the inner lumen of an introducer catheter. The wire guide is advanced out of the distal end of the introducer catheter into the patient until the distal end of the wire guide reaches the location where the interventional procedure is to be performed. After the wire guide is inserted, another device such as a stent and stent delivery catheter is advanced over the previously introduced wire guide into the patient until the stent delivery catheter is in the desired location. After the stent has been delivered, the stent delivery catheter can then be removed from a patient by retracting the stent delivery catheter back over the wire guide. The wire guide may be left in place after the procedure is completed to ensure easy access if it is required.

Conventional wire guides include an elongated wire core with one or more tapered sections near the distal end to increase flexibility. Generally, a flexible body such as a helical coil or tubular body is disposed about the wire core. The wire core is secured to the flexible body at the distal end. In addition, a torquing means can be provided on the proximal end of the core member to rotate, and thereby steer a wire guide having a curved tip, as it is being advanced through a patient's vascular system.

A major requirement for wire guides and other intraluminal guiding members is that they have sufficient stiffness to be pushed through the patient's vascular system or other body lumen without kinking. However, they must also be flexible enough to pass through the tortuous passageways without damaging the blood vessel or any other body lumen through which they are advanced. Efforts have been made to improve both the strength and the flexibility of wire guides to make them more suitable for their intended uses, but these two properties tend to be diametrically opposed to one another in that an increase in one usually involves a decrease in the other.

For certain procedures, such as when delivering stents around challenging take-off, tortuosities, or severe angulation, substantially more support and/or vessel straightening is frequently needed from the wire guide. Wire guides have been commercially available for such procedures which provide improved support over conventional wire guides. However, such wire guides are in some instances are so stiff they can damage vessel linings when being advanced.

In other instances, extreme flexibility is required as well. For example, when branched or looped stents are to be delivered to a branched vascular region, it is beneficial to insert the wire guide from the branch where a stent is to be located. However, the stent may need to be introduced and guided from a separate branch. In this situation, the wire guide is inserted into the patient's vascular system near the desired stent location and a grasping device is inserted in the branch from which the stent will be introduced. The wire guide may be advanced back along the branch to provide the grasping device access to the distal end of the wire guide. However, the wire guide should be extremely flexible to allow grasping and manipulation of the wire guide without damaging the tissue around the bifurcation formed by the luminal branch. Further, the wire guide should be extremely kink resistant to avoid damaging the wire guide as it is grasped. After the wire guide is retrieved by the grasping device, the stent may be delivered over the wire guide to the desired location. However, available wire guides are not designed to provide the flexibility required to cross up and over the bifurcation of the luminal branch and yet also provide the stiffness required to aid in the insertion of the stent.

In view of the above, it is apparent that there exists a need for an improved design for a wire guide.

### BRIEF SUMMARY

One aspect provides a wire guide having variable flexibility along its length. In one embodiment, the wire guide includes a multi-filar coil having a proximal end and a distal end and having an increasing pitch towards the distal end. The proximal portion of a core member is positioned within the multi-filar coil. In another embodiment the distal end of the multi-filar coil is attached to the core member.

In one embodiment, the multi-filar coil includes at least 6 and not more than 12 filars. In another embodiment, the filars within the multifilar coil are arranged in at least 2 layers.

In yet another embodiment, the pitch of the multifilar coil increases by at least 25 percentage from the proximal end to the distal end. In another embodiment, the pitch of the multifilar coil increases by at least 50 percentage from the proximal end to the distal end.

In various embodiments, the multifilar coil includes stainless steel, tantalum, a nickel-titanium alloy, gold, silver, tungsten, palladium, platinum, a cobalt-chromium alloy, iridium or combinations thereof. In other embodiments, the core member includes stainless steel, tantalum, a nickel-titanium alloy, gold, silver, tungsten, platinum, a cobalt-chromium alloy, iridium or combinations thereof.

### BRIEF DESCRIPTION OF THE DRAWINGS

FIG. 1 shows an exploded view of the distal portion of one embodiment of a wire guide.

FIG. 2 shows a view of the distal portion of one embodiment of a wire guide.

FIG. 3 shows a view of the distal portion of another embodiment of a wire guide.

FIG. 4 shows a sectional view of a multi-filar coil having two layers.

### DETAILED DESCRIPTION

One aspect provides a wire guide that has variable flexibility along its length. In one embodiment, the flexibility of at least a portion of the wire guide increases towards the distal end of the wire guide. As used herein, the term “proximal” refers to a portion of the wire guide closest to a physician when placing a wire guide in the patient, and the term “distal” refers to a portion of the wire guide closest to the end inserted into the patient's body. In one embodiment the wire guide includes a multi-filar coil having an increasing flexibility towards the distal end of the wire guide.

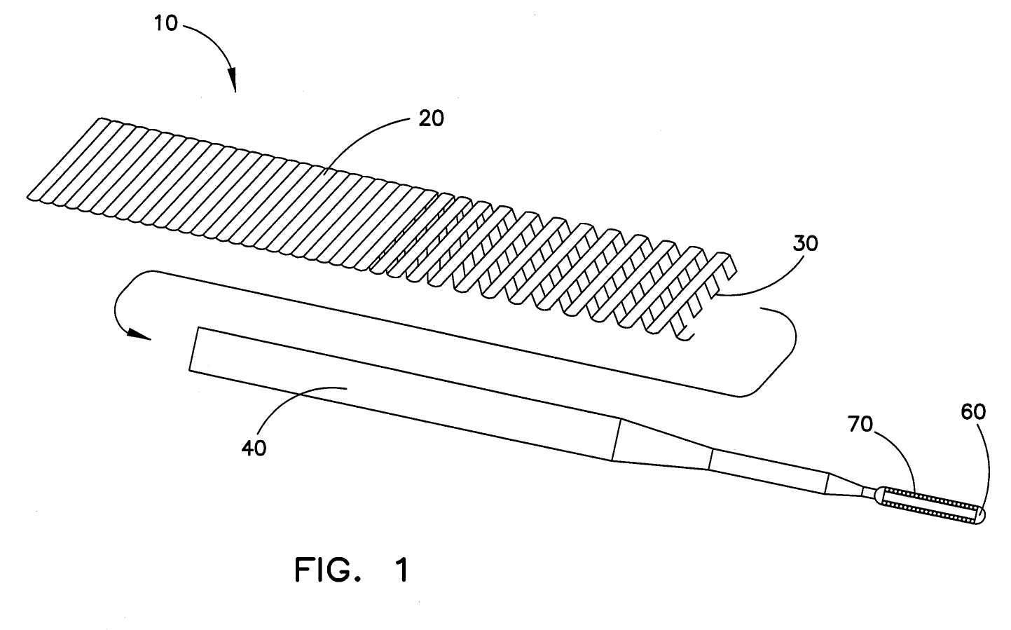

Referring now to FIG. 1, this figure illustrates an exploded view of the distal portion of wire guide 10 incorporating hollow multi-filar coil 20 and core member 40. In one embodiment, multi-filar coil 20 is placed over the proximal portion of core member 40 and extends from near the proximal end of wire guide 10 towards the distal end 60 of wire guide 10. In one embodiment, distal end 30 of multi-filar coil 20 is attached to core member 40. Attachment may be by any suitable means including, but not limited to, soldering, welding, or by adhesive. In other embodiments, distal end 30 of multi-filar coil 20 is not attached to core member 40. In this embodiment, multi-filar coil 20 may be moved axially along core member 40.

In certain embodiments, wire guide 10 includes a second coil 70 positioned near distal end 60 of core member 40. In other embodiments, second coil 70 is not present. In certain embodiments, core member 40 has a substantially constant cross sectional dimension along its length. In other embodiments, core member 40 includes one or more tapers. For example, core member 40 may include one or more tapers reducing its cross sectional dimension towards the distal end of the wire guide relative to that of the proximal portion.

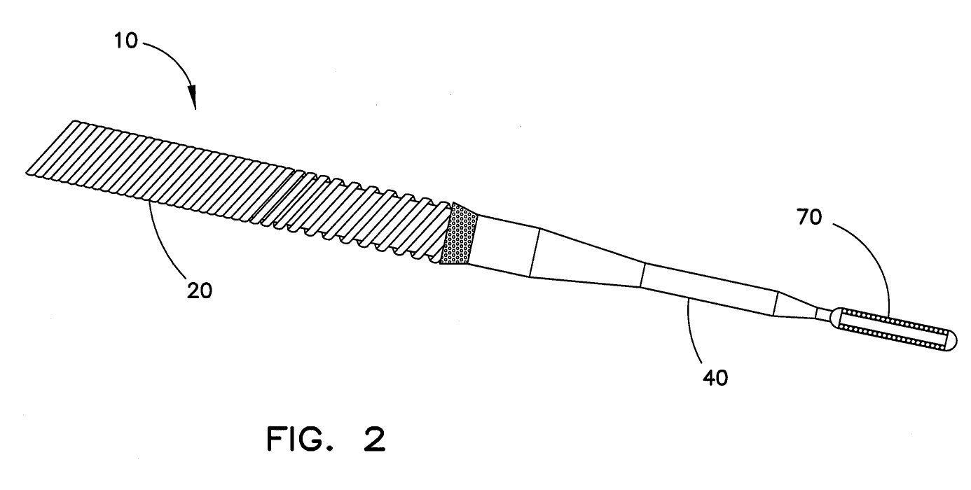

Referring now to FIG. 2, this figure illustrates an assembled view of the distal portion of wire guide 10. A proximal portion of core member 40 is shown positioned within the lumen of multi-filar coil 20. In one embodiment, the distal end of multi-filar coil 20 is attached to core member 40. In another embodiment, the distal end of multi-filar coil 20 is not attached to core member 40. In certain embodiments, second coil 70 is positioned at the distal end of core member 40.

In one embodiment, the wire guide is constructed such that the multi-filar coil have be moved proximally or a distally along the core member. This configuration is advantageous in that it allows the flexibility of the wire guide to be varied while the wire guide is partially inserted within a body lumen. For example, it situations where extreme flexibility is required, such as when the wire guide must pass through a tortuous passageway without damaging a blood vessel, the multi-filar coil is moved proximally with respect to the core member, resulting in an increase in the flexibility of the distal region of the wire guide. In other situations, for example then more stiffness in required to aid passage of the wire guide, the multi-filar coil is moved distally with respect to the core member, resulting in a decrease in the flexibility of the distal region of the wire guide. In certain embodiments, the wire guide also includes a locking mechanism, such as an Olcott or Hemostat lock (not illustrated), to allow the relative axial positions of multi-filar coil and core member to be fixed.

The wire guide may have typical wire guide dimensions. In certain embodiments, the wire guide length is about 90 to about 300 cm, and for use within a patient's coronary system available wire guides are typically about 180 cm in length. In one embodiment, the outside diameter of the multi-filar coil is between 0.010 inches and 0.090 inches.

In certain embodiments, the core element is manufactured from a material such as stainless steel, a stainless steel alloy, a nickel-titanium alloy, such as nitinol, or combinations of these materials. Inclusion of a radiopaque material, such as platinum or gold, as part of the core element allows for better visibility during manipulation of the wire guide within the body of a patient. In certain embodiments, a radiopaque material is included in other portions of the wire guide, for example, as part of the multi-filar coil.

In various embodiments, multi-filar coil 20 is formed from materials including, but not limited to, stainless steel, alloys including stainless steel, nickel-titanium alloys, such as NITINOL®, or combinations of these materials. In one embodiment, the multi-filar coil includes between 3 and 15 filars. In other embodiments, there are between 4, 5, 6, 7, 8, 9, 10, 11 or 12 and 15 filars. In yet other embodiments, there are between 3 and 6, 7, 8, 9, 10, 11, 12, 13, 14 and 15 filars. In other embodiments, there are more than 15 filars. In various embodiments, the filars form helical hollow strands. In one embodiment, each of the filars is formed from the same material. In other embodiments, the filars are formed for different materials. For example, some filars are formed from stainless steel and others from NITINOL®. The filars may be of the same cross sectional dimension or may be of differing cross sectional dimension.

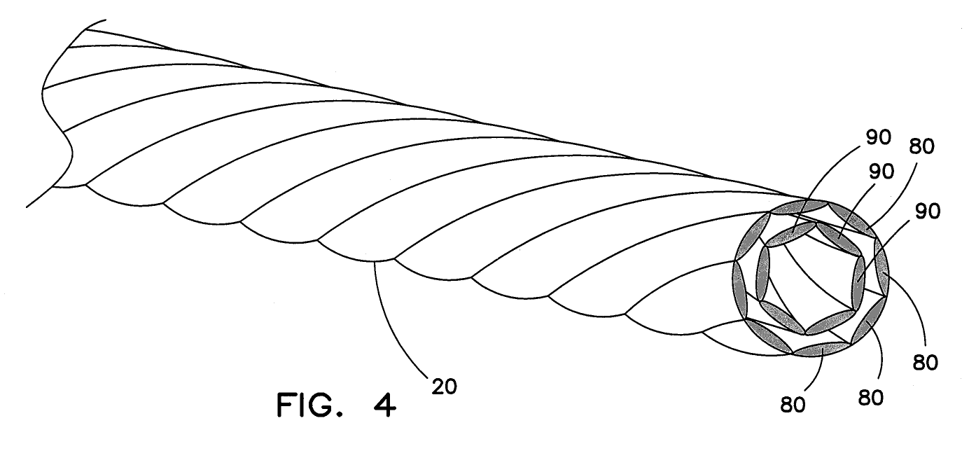

In one embodiment, the filars are arranged in a single layer within the coil. In another embodiment, the filars are arranged in two layers within the coil. FIG. 4 illustrates a coil having filar arranged in two layers. The outer layer is formed of 8 filars 80 and the inner layer is formed from 6 filars 90. In other embodiments, the filars are arranged in 3 or 4 layers.

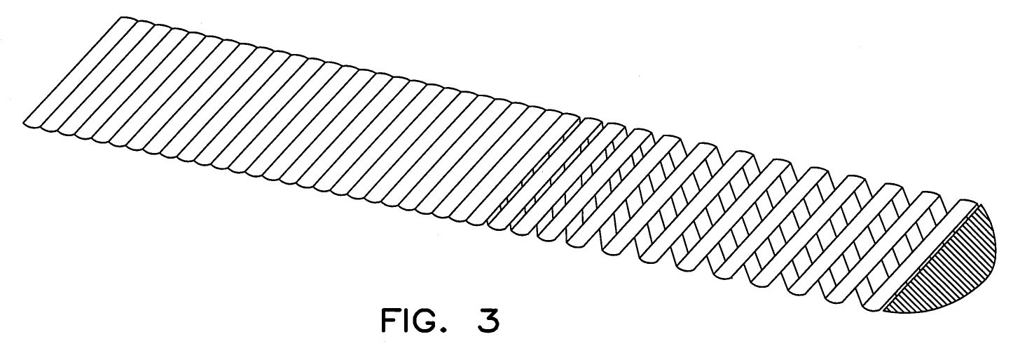

In yet another embodiment, illustrated in FIG. 3, the device does not include a core member. In this embodiment, the device includes a multi-filar coil extending from the proximal to the distal end of the device. The multi-filar coil may include multiple layers formed from the same or differing materials. In one embodiment, the pitch of the filars in one or more of the layers increases towards the distal end of the device, resulting in increased flexibility towards the distal end.

For the purposes for the present description the pitch of a helical strand is the length of one complete helix turn of the strand, measured along the axis of the helical strand. In certain embodiments, the pitch of the filars of the helical hollow strands is constant along the length of the multi-filar coil. In other embodiments, the pitch of the helical hollow strands varies along the length of the multi-filar coil. For example, in certain embodiments the pitch of filars increases towards the distal end of the multi-filar coil. In one embodiment, increasing the pitch of the coil towards the distal end of the coil result in the distal portion of the coil having a greater flexibility that the proximal portion.

In one embodiment, the pitch of the filars increases by 10% along the length from the proximal end to the distal end of the coil. In other embodiments, the pitch of the filars increases by 20%, 30%, 40%, 50%, 60%, 70%, 80%, 90%, 100%, 120%, 140%, 160%, 180%, 200%, 250% or 300% along the length from the proximal end to the distal end of the coil. In one embodiment, the rate of increase in pitch of the filars is constant along the length of the coil. In other embodiments, the rate of increase in pitch of the filars varies along the length of the coil.

In certain embodiments, the wire guide further includes a coating on at least a portion of the outer surface of multi-filar coil and or the core member. The coating can include a material that reduces the coefficient of friction on that surface. For example, the coating may include a polymer such as, but not limited to, a fluoropolymer.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

I claim:

1. A wire guide comprising:
a multi-filar coil having a proximal end and a distal end and having an increasing pitch forwards the distal end; and
a core member having a proximal portion and a distal portion, where the proximal portion of the core member in positioned within the multi-filar coil.

2. The wire guide of claim 1, wherein the distal end of the multi-filar coil is attached to the core member.

3. The wire guide of claim 1, wherein the multi-filar coil comprises at least 6 and not more than 12 filars.

4. The wire guide of claim 1, wherein the multi-filar coil comprises at least 2 layers.

5. The wire guide of claim 1, wherein the pitch of the multi-filar coil increases by at least 25 percentage from the proximal end to the distal end.

6. The wire guide of claim 5, wherein the pitch of the multi-filar coil increases by at least 50 percentage from the proximal end to the distal end.

7. The wire guide of claim 1, wherein the multi-filar coil comprises a material selected from the group consisting of stainless steel, tantalum, a nickel-titanium alloy, gold, silver, tungsten, palladium, platinum, a cobalt-chromium alloy, iridium and combinations thereof.

8. The wire guide of claim 7, wherein the multi-filar coil comprises a nickel-titanium alloy.

9. The wire guide of claim 1, wherein the core member comprises a taper from a proximal end to a distal end.

10. The wire guide of claim 1, wherein the core member comprises a material selected from the group consisting of stainless steel, tantalum, a nickel-titanium alloy, gold, silver, tungsten, platinum, a cobalt-chromium alloy, iridium and combinations thereof.

11. The wire guide of claim 10, wherein the core member comprises a nickel-titanium alloy

12. The wire guide of claim 1, wherein the multi-filar coil is slidably adjustable along the core member.

13. The wire guide of claim 1, further comprising a second coil positioned at a distal end of the core member.

14. The wire guide of claim 13, wherein the second coil comprises platinum or palladium.

15. The wire guide of claim 1, wherein the outside diameter of the multi-filar coil is between 0.010 inches and 0.090 inches.
